(19) United States
(12) Patent Application Publication
(10) Pub. No.: US 2026/0318481 A1
(43) Pub. Date: Oct. 15, 2026

(54) METHOD FOR PRODUCING ACID FLUORIDE-FILLED CONTAINER

(71) Applicant: c/o Resonac Corporation, Tokyo (JP)

(72) Inventor: Kazuma MATSUI, Tokyo (JP)

(73) Assignee: Resonac Corporation, Tokyo (JP)

(21) Appl. No.: 19/480,173

(22) PCT Filed: Nov. 7, 2024
(86) PCT No.: PCT/JP2024/039703

§ 371 Date: Oct. 30, 2025

(30) Foreign Application Priority Data
Nov. 13, 2023 (JP) 2023-193030

(51) Int. Cl. F17C 5/00; C23F 11/02

(52) U.S. Cl. CPC F17C 5/00; C23F 11/02; F17C 2203/0639; F17C 2221/03; F17C 2260/044

(57) ABSTRACT

A method for producing an acid fluoride-filled container includes filling a container with an acid fluoride, where the container has at least an inner surface formed of a metal material and has a surface roughness Rz of the inner surface of 0.01 μm or more and 10 μm or less. The method includes: a treatment step of introducing a treatment gas containing an acid fluoride for treatment for treating the inner surface, into the container to bring the inner surface into contact with the acid fluoride for treatment at a temperature of 160° C. or less; an extraction step of extracting the treatment gas introduced into the container in the treatment step, from the container; and a filling step of filling the container after the extraction step with an acid fluoride for being stored in the container.

### TECHNICAL FIELD

The present disclosure relates to a method for producing an acid fluoride-filled container.

### BACKGROUND ART

As a container in which an acid fluoride is filled and stored, a container made of a metal material is often used. However, when the acid fluoride is stored in a container made of a metal material, there is a case where the acid fluoride reacts with a metal material or carbon forming an inner surface of a container made of a metal material, or there is a case where the acid fluoride reacts with water attached to an inner surface of a container made of a metal material. As a result, gas impurities such as carbon monoxide (CO), carbon dioxide (CO₂), carbon tetrafluoride (CF₄), and hydrogen fluoride (HF), and metal impurities are generated, and thus there is a concern that such a problem that the purity of the acid fluoride stored in a container made of a metal material is gradually decreased may occur.

### CITATION LIST

### Patent Literature

PTL 1: JP 2009-57248 A

### SUMMARY OF INVENTION

### Technical Problem

An object of the present disclosure is to provide a method for producing an acid fluoride-filled container, where an acid fluoride filled into the acid fluoride-filled container is less likely to cause a decrease in purity.

### Solution to Problem

To achieve the above object, one aspect of the present disclosure is as the following [1] to [6].
[1] A method for producing an acid fluoride-filled container in which an acid fluoride is filled into a container, in which at least an inner surface of the container is formed of a metal material and a surface roughness Rz of the inner surface is 0.01 μm or more and 10 μm or less, and the method including:
a treatment step of introducing a treatment gas containing an acid fluoride for treatment for treating the inner surface, into the container to bring the inner surface into contact with the acid fluoride for treatment at a temperature of 160° C. or less;
an extraction step of extracting the treatment gas introduced into the container in the treatment step, from the container; and
a filling step of filling the container after the extraction step with an acid fluoride for being stored in the container.
[2] The method for producing an acid fluoride-filled container according to [1], in which in the metal material forming the inner surface, an iron content is 60% by mass or more, and a chromium content is 10% by mass or less.
[3] The method for producing an acid fluoride-filled container according to [1], in which the metal material forming the inner surface is at least one of manganese steel and chromium-molybdenum steel.
[4] The method for producing an acid fluoride-filled container according to any one of [1] to [3], in which a content of any of carbon monoxide, carbon dioxide, carbon tetrafluoride, and hydrogen fluoride in the acid fluoride extracted from the container after the filling step is 10 ppm by volume or less.
[5] The method for producing an acid fluoride-filled container according to any one of [1] to [4], in which a content of any metal contained in a liquid phase of the acid fluoride extracted from the container after the filling step is less than 100 ppb by mass for any metal.
[6] The method for producing an acid fluoride-filled container according to any one of [1] to [5], in which the acid fluoride filled into the container in the filling step is at least one of carbonyl fluoride, oxalyl fluoride, and trifluoromethyl acetyl fluoride.

### Advantageous Effects of Invention

According to the method for producing an acid fluoride-filled container according to the present disclosure, it is possible to produce an acid fluoride-filled container, where an acid fluoride filled into the acid fluoride-filled container is less likely to cause a decrease in purity.

### BRIEF DESCRIPTION OF DRAWINGS

FIG. 1 is a schematic view of an acid fluoride filling device for describing a method for producing an acid fluoride-filled container according to one embodiment of the present disclosure.

### DESCRIPTION OF EMBODIMENTS

One embodiment of the present disclosure will now be described. The embodiment is merely one example of the present disclosure, and the present disclosure is not limited to the embodiment. Various modifications or improvements can be made in the embodiment, and such modifications and improvements can be encompassed by the present disclosure. In the present specification, the pressure is indicated in terms of gauge pressure unless otherwise specified.

A method for producing an acid fluoride-filled container according to the present embodiment is a method for producing an acid fluoride-filled container in which an acid fluoride is filled into a container, in which at least an inner surface of the container is formed of a metal material and a surface roughness Rz of the inner surface is 0.01 μm or more and 10 μm or less, and the method including:
a treatment step of introducing a treatment gas containing an acid fluoride for treatment for treating the inner surface, into the container to bring the inner surface into contact with the acid fluoride for treatment at a temperature of 160° C. or less;
an extraction step of extracting the treatment gas introduced into the container in the treatment step, from the container; and
a filling step of filling the container after the extraction step with an acid fluoride for being stored in the container.

In the acid fluoride-filled container produced by the method for producing an acid fluoride-filled container according to the present embodiment, an acid fluoride filled into the acid fluoride-filled container is less likely to cause a decrease in purity.

Therefore, the acid fluoride-filled container produced by the method for producing an acid fluoride-filled container according to the present embodiment, and the acid fluoride filled into the acid fluoride-filled container is capable of being used as an electronic material gas for manufacturing a semiconductor or a raw material for precise organic synthesis.

### [Acid Fluoride Filling Device]

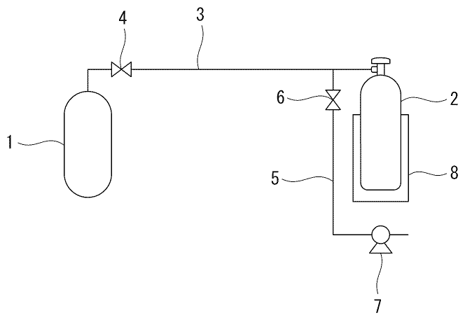

An example of a configuration of an acid fluoride filling device that is capable of being used in the method for producing an acid fluoride-filled container according to the present embodiment will be described with reference to FIG. 1.

The acid fluoride filling device of FIG. 1 includes a raw material tank 1 accommodated an acid fluoride, a cylinder 2 that is a container for storing an acid fluoride, a pipe 3 connected the raw material tank 1 to the cylinder 2, and a valve 4 provided in the pipe 3. At least the inner surface of the cylinder 2 is formed of a metal material (for example, a cylinder made of a metal material), and the surface roughness Rz of the inner surface is 0.01 μm or more and 10 μm or less.

A branch pipe 5 is branched to extend from a portion of the pipe 3 on a downstream side of the valve 4, the branch pipe 5 is connected to a vacuum pump 7, and an exhaust port of the vacuum pump 7 is connected to a detoxifying device (not illustrated). A valve 6 is provided in a portion of the branch pipe 5 on the upstream side of the vacuum pump 7. Further, the cylinder 2 is equipped with a temperature controller 8, and thus the temperature of the cylinder 2 can be set to any temperature.

By using an acid fluoride filling device having such a configuration, the method for producing an acid fluoride-filled container according to the present embodiment can be carried out to produce an acid fluoride-filled container. More specifically, the acid fluoride accommodated in the raw material tank 1 is introduced into the cylinder 2 through the pipe 3, the acid fluoride is brought into contact with the inner surface of the cylinder 2 at any temperature of 160° C. or less (treatment step), and then the acid fluoride is discharged from the cylinder 2 through the branch pipe 5 by using the vacuum pump 7 (extraction step). Then, the acid fluoride accommodated in the raw material tank 1 is introduced again into the cylinder 2 through the pipe 3 (filling step), whereby it is possible to produce an acid fluoride-filled acid fluoride-filled container having a small amount of impurities.

The acid fluoride filling device may include a device that is not illustrated in FIG. 1. For example, a regulator for filling the cylinder 2 with the acid fluoride accommodated in the raw material tank 1 at any pressure, or a mass flow controller for controlling a flow rate of the acid fluoride when the acid fluoride accommodated in the raw material tank 1 is sent to the cylinder 2 may be provided.

### [Acid Fluoride]

The acid fluoride is a compound having a functional group of *—C(═O)F in the molecule. “*” means any atom or atomic group. The number of carbon atoms contained in the acid fluoride is preferably 5 or less, more preferably 3 or less, and still more preferably 2 or less.

Examples of the acid fluoride include carbonyl fluoride (COF₂), oxalyl fluoride (C₂O₂F₂), trifluoromethyl acetyl fluoride (CF₃CH₂COF), formyl fluoride, carbonyl chloride fluoride, acetyl fluoride, 2,2,3,3,3-pentafluoropropanoyl fluoride, 2,2,3,3,4,4,4-heptafluorobutanoyl fluoride, 2,2,3,3,4,4,5,5,5-nonafluoropentanoyl fluoride, 2,2,3,4,4,4-hexafluoro-3-(trifluoromethyl)butanoyl fluoride, and 3,3,3-trifluoro-2,2-bis(trifluoromethyl)propanoyl fluoride.

Among these acid fluorides, carbonyl fluoride, oxalyl fluoride, or trifluoromethyl acetyl fluoride is preferable, and carbonyl fluoride is more preferable from the viewpoint of ease of availability. One type of the acid fluoride may be used alone, or two or more types thereof may be used in combination.

More specifically, the acid fluoride to be filled into the container in the filling step may be at least one of carbonyl fluoride, oxalyl fluoride, and trifluoromethyl acetyl fluoride.

The acid fluoride to be filled into the container in the filling step preferably has high purity. However, excessive purification leads to an increase in the manufacturing cost of the acid fluoride-filled container, and thus the acid fluoride may contain impurities as long as the amount thereof is small.

For example, the purity of the acid fluoride to be filled into the container in the filling step is preferably 95% by volume or more and 100% by volume or less, more preferably 98% by volume or more and 99.99999% by volume or less, and still more preferably 99% by volume or more and 99.9999% by volume or less. When the purity of the acid fluoride to be filled into the container in the filling step is within the above-described numerical value range, the acid fluoride filled into the acid fluoride-filled container is less likely to be mixed with gas impurities and metal impurities, and thus a decrease in purity is less likely to occur.

### [Impurities]

In the present disclosure, the impurities refer to components other than the components intentionally mixed with the acid fluoride filled into the acid fluoride-filled container. Examples of impurities that may be contained in the acid fluoride filled into the acid fluoride-filled container include gas impurities such as a nitrogen gas (N₂), a hydrogen gas (H₂), carbon dioxide (CO₂), an oxygen gas (O₂), argon (Ar), water (H₂O), hydrogen fluoride (HF), carbon monoxide (CO), and carbon tetrafluoride (CF₄), and metal impurities.

Among the gas impurities, carbon monoxide, carbon dioxide, hydrogen fluoride, or carbon tetrafluoride may be generated during storage when the inner surface of the container filled with the acid fluoride is formed of a metal material. The content of gas impurities contained in the acid fluoride filled into the acid fluoride-filled container is preferably 5% by volume or less, more preferably 1% by volume or less, still more preferably 300 ppm by volume or less, even still more preferably 100 ppm by volume or less, even further still more preferably 10 ppm by volume or less, and particularly preferably 5 ppm by volume or less, regardless of the type of gas impurities.

For example, it is preferable that a content of any of carbon monoxide, carbon dioxide, carbon tetrafluoride, and hydrogen fluoride in the acid fluoride extracted from the container after the filling step is 10 ppm by volume or less.

In addition, when an acid fluoride containing a metal is used for etching, the inside of the chamber may be contaminated with a metal, there is a concern that which causes non-uniformity of plasma due to abnormal discharge and metal contamination of an etching target object. Therefore, it is preferable to remove the metal as much as possible by purification.

Regarding the type of the metal, for example, metal elements of the third to sixth periods of the periodic table correspond to the metal, and examples thereof include Na, Mg, Al, K, Ca, Cr, Mn, Fe, Co, Ni, Cu, Zn, Sb, Mo, and W. These metals may be contained in the acid fluoride as a simple body and/or a metal compound.

The metal compound refers to a compound having a metal element and a non-metal element, and examples thereof include a metal oxide, a metal nitride, a metal oxynitride, a metal chloride, a metal bromide, a metal iodide, and a metal sulfide.

Among the above-described metals, Na, Cr, Mn, Fe, Co, Ni, Cu, and Mo are often contained in a container or a metal pipe, which is filled with an acid fluoride. Therefore, these metals are likely to be mixed into the acid fluoride. In particular, Cr reacts with the acid fluoride to form a volatile metal complex (CrO₂F₂, CrF₆, or the like), and thus Cr is likely to be mixed into the acid fluoride.

The content of the metal in the acid fluoride can be quantified, for example, by using a method such as inductively coupled plasma mass spectrometry (ICP-MS).

The content of the metal contained in the acid fluoride filled into the acid fluoride-filled container is preferably 0.1 ppb by mass or more and 300 ppb by mass or less, more preferably 0.5 ppb by mass or more and 100 ppb by mass or less, and still more preferably 1 ppb by mass or more and 20 ppb by mass or less, regardless of the type of metal.

For example, it is preferable that a content of any metal contained in a liquid phase of the acid fluoride extracted from the container after the filling step is less than 100 mass ppb for any metal.

### [Material of Container]

Regarding a container for producing an acid fluoride-filled container by filling the container with an acid fluoride, the material of the container is not particularly limited as long as at least the inner surface is formed of a metal material and a surface roughness Rz of the inner surface is 0.01 μm or more and 10 μm or less. However, a metal container formed of entirely of a metal material is preferable.

The type of the metal material is not particularly limited, and a metal material having an iron content of 60% by mass or more and a chromium content of 10% by mass or less is preferable. More specifically, it is preferable that, in the metal material forming the inner surface of the container, the iron content is 60% by mass or more, and the chromium content is 10% by mass or less.

Although the type of the metal material is not particularly limited, manganese steel, chromium-molybdenum steel, stainless steel, or the like is preferable. More specifically, the metal material forming the inner surface of the container is preferably at least one of manganese steel and chromium-molybdenum steel.

It is preferable that the manganese steel contains 97% by mass or more of iron and contains 1% by mass or more and 2% by mass or less of manganese. When carbon or chromium is contained in the manganese steel, the content of carbon is preferably 0.5% by mass or less, and the content of chromium is preferably 0.35% by mass or less. Examples of the manganese steel include SMn420, SMn433, SMn438, SMn443, STH11, and STH12.

It is preferable that the chromium-molybdenum steel contains 95% by mass or more of iron and contains 0.9% by mass or more and 1.5% by mass or less of chromium. When carbon is contained in the chromium-molybdenum steel, the content of carbon is preferably 0.5% by mass or less. Examples of the chromium-molybdenum steel include STH21 and STH22.

The stainless steel contains 70% by mass or more of iron and may contain 10.5% by mass or more of chromium. When the container made of stainless steel is filled with an acid fluoride, as described above, chromium reacts with the acid fluoride to form a volatile metal complex, and thus chromium is likely to be contained in the acid fluoride gas. Therefore, it is preferable that the content of chromium in the stainless steel is usually less than 5% by mass. However, when the acid fluoride-filled container is produced by the method according to the present disclosure, the mixing of chromium into the acid fluoride is suppressed even when the container made of stainless steel is filled with the acid fluoride.

When carbon is contained in the stainless steel, the content of carbon is preferably 1.5% by mass or less.

Examples of the stainless steel include SUS304, SUS316, and SUS316L.

### [Surface Roughness Rz of Inner Surface of Container]

Regarding a container for producing an acid fluoride-filled container by filling the container with an acid fluoride, the surface roughness Rz of the inner surface is preferably 0.01 μm or more and 10 μm or less, more preferably 0.05 μm or more and 5 μm less, and particularly preferably 0.1 μm or more and 2 μm or less. The surface roughness Rz means a maximum height roughness defined in JIS B0601: 2001.

When the surface roughness Rz of the inner surface of the container is small, the gas adsorption performance of the inner surface of the container is lowered. Therefore, the amount of water that is adsorbed and remains on the inner surface of the container is reduced, and thus the content of water mixed into the acid fluoride is likely to be low. In addition, the amount of carbon monoxide, carbon dioxide, carbon tetrafluoride, and hydrogen fluoride, which are generated by the reaction between the acid fluoride and water, is small, and thus the purity of the acid fluoride filled into the container is less likely to decrease.

In addition, when the surface roughness Rz of the inner surface of the container is small, the area for contact with the acid fluoride is small, and thus a reaction between the metal material and the acid fluoride is less likely to occur, and the metal is less likely to be released into the acid fluoride.

Therefore, by reducing the surface roughness Rz of the inner surface of the container, the amount of water adsorbed on the inner surface of the container is reduced, and the reaction between the metal material and the acid fluoride is suppressed, and thus the purity of the acid fluoride filled into the container is less likely to decrease.

The surface roughness Rz of the inner surface of the container can be adjusted by polishing, for example. The polishing method is not particularly limited, and for example, electropolishing, buff polishing, barrel polishing, or the like can be used.

### [Treatment Step]

The method for producing an acid fluoride-filled container according to the present embodiment has a treatment step of introducing a treatment gas containing an acid fluoride for treatment for treating an inner surface of a container, into the container to bring the inner surface of the container into contact with the acid fluoride for treatment at a temperature of 160° C. or less.

In the treatment step, the water adsorbed on the inner surface of the container is allowed to react with the acid fluoride for treatment, and the acid fluoride for treatment is allowed to react with impurities such as chromium and carbon on the inner surface of the container, whereby carbon monoxide, carbon dioxide, carbon tetrafluoride, and hydrogen fluoride are generated, and the impurities are volatilized.

The treatment gas may be a gas consisting of only the acid fluoride for treatment or may be a mixed gas of the acid fluoride for treatment and another type of gas. Examples of the other type of gases include an inert gas such as a nitrogen gas or argon.

The acid fluoride for treatment that is used in the treatment step and the acid fluoride that is stored in the container in the filling step may be acid fluorides of the same type or may be acid fluorides of different types.

When the temperature of the treatment step is 160° C. or less, the generation of the carbon monoxide or fluorine gas (F₂) due to the decomposition of the acid fluoride for treatment is less likely to occur. However, when the temperature of the treatment step is too low, the reaction between the acid fluoride for treatment and impurities is less likely to proceed, and thus the temperature of the treatment step is preferably −78° C. or more and 160° C. or less, more preferably −30° C. or more and 100° C. or less, still more preferably 10° C. or more and 80° C. or less, and particularly preferably more than 40° C. and 75° C. or less.

The pressure of the treatment step is not particularly limited, and can be set appropriately, for example, in a range of 10 kPa or more and 15 MPa or less. The treatment step may be carried out in a state where the acid fluoride for treatment is vaporized in the container. However, it is desirable to carry out the treatment step at a pressure or a critical pressure at which the acid fluoride for treatment is liquefied in the container. Specifically, the pressure in the container is preferably 0.3 MPa or more and 10 MPa or less, more preferably 1 MPa or more and 9 MPa or less, still more preferably 1.5 MPa or more and 8 MPa or less, and particularly preferably 6 MPa or more and 7.6 MPa or less. The state of the phase change of the acid fluoride for treatment in the container can be controlled by the temperature and pressure in the treatment step, and the filling amount of the acid fluoride for treatment.

The time for carrying out the treatment step is not particularly limited as long as a time during which the acid fluoride for treatment sufficiently reacts with impurities in the container can be ensured, and the time may be set to 20 seconds or more and 300 hours or less, for example. In consideration of the reproducibility and productivity of the treatment step, the timer for the treatment step is preferably 1 minute or more and 200 hours or less, more preferably 5 minutes or more and 100 hours or less, still more preferably 10 minutes or more and 48 hours or less, and particularly preferably 15 minutes or more and 24 hours or less. When the time for carrying out the treatment step is within the above-described range, the impurities in the container can be allowed to sufficiently react with the acid fluoride for treatment.

The treatment step may be carried out in a state where the container is allowed to stand, or may be carried out while moving the container using a cylinder rotary machine or a shaker. When the treatment step is carried out while moving the container, the acid fluoride for treatment causes convection in the container, which makes it possible to improve the efficiency of the reaction between the acid fluoride for treatment and the impurities. In addition, the treatment step may be carried out only once or may be repeatedly carried out a plurality of times. When the treatment step is repeatedly carried out a plurality of times, the treatment step is carried out to be followed by an extraction step described later, and then the next treatment step is carried out.

### [Extraction Step]

The method for producing an acid fluoride-filled container according to the present embodiment has an extraction step of extracting the treatment gas introduced into the container in the treatment step, from the container. In the extraction step, the entire amount of the treatment gas introduced into the container may be extracted, or a part of the treatment gas introduced into the container may be extracted. When a part of the treatment gas introduced into the container is extracted, the amount of the treatment gas to be extracted may be determined so that the amount of impurities contained in the treatment gas remaining in the container is reduced to be equal to or smaller than the reference value.

From the viewpoint of suppressing the amount of the treatment gas to be used, it is preferable that the amount of the treatment gas to be extracted from the container is small. For example, the amount of the treatment gas to be extracted may be 0.1% by mass or more and 100% by mass or less of the amount of the treatment gas introduced into the container in the treatment step. However, it is preferably 3% by mass or more and 90% by mass or less and more preferably 10% by mass or more and 80% by mass or less.

### [Filling Step]

The method for producing an acid fluoride-filled container according to the present embodiment has a filling step of filling the container after the extraction step with an acid fluoride for being stored in the container. The amount of the acid fluoride to be filled in the filling step is not particularly limited as long as it is an amount that does not cause overfilling of the container. The overfilling refers to that the inside of the container is completely filled with the liquefied acid fluoride.

The temperature at which the filling step is carried out is not particularly limited, and is preferably higher than the temperature at which the acid fluoride is solidified. In addition, the amount of impurities derived from the container is likely to be reduced when the filling step is carried out at a temperature lower than the temperature of the treatment step, and thus it is preferable that the temperature at which the filling step is carried out is a temperature lower than the temperature of the treatment step.

For example, the temperature at which the filling step is carried out is preferably −84° C. or more and 120° C. or less, more preferably −79° C. or more and 80° C. or less, still more preferably −20° C. or more and 60° C. or less, and particularly preferably 0° C. or more and 30° C. or less.

In the acid fluoride-filled container produced by the method for producing an acid fluoride-filled container according to the present embodiment, the mixing of gas impurities and metal impurities into the filled acid fluoride is less likely to occur, and thus the acid fluoride filled into the acid fluoride-filled container is less likely to cause a decrease in purity. The purity of the acid fluoride extracted from the acid fluoride-filled container produced by the method for producing an acid fluoride-filled container according to the present embodiment is preferably 99% by volume or more, and more preferably 99.9% by volume or more.

A method of measuring the purity of the acid fluoride is not particularly limited, and the purity thereof is capable of being determined by analyzing the content of impurities such as carbon monoxide, carbon dioxide, carbon tetrafluoride, hydrogen fluoride, and metals in the acid fluoride, by using Fourier transform infrared spectroscopy (FT-IR), inductively coupled plasma mass spectrometry (ICP-MS), or the like.

### EXAMPLES

The present disclosure will be described in more detail below with reference to Examples and Comparative Examples.

### Example 1

Using a filling device having substantially the same configuration as the acid fluoride filling device illustrated in FIG. 1, an acid fluoride-filled container was produced (see Table 1). Both the acid fluoride for treatment and the acid fluoride for storage in a container are carbonyl fluoride, and they are accommodated in a raw material tank.

Gas impurities and metal impurities, which are contained in the carbonyl fluoride, are as follows. More specifically, the content of carbon monoxide is 5 ppm by volume, the content of carbon dioxide is 6 ppm by volume, the content of hydrogen fluoride is 4 ppm by volume, the content of carbon tetrafluoride is 3 ppm by volume, the content of sodium is less than 5 ppb by mass, the content of chromium is less than 5 ppb by mass, the content of manganese is less than 5 ppb by mass, the content of iron is less than 5 ppb by mass, the content of cobalt is less than 5 ppb by mass, the content of nickel is less than 5 ppb by mass, the content of copper is less than 5 ppb by mass, and the content of molybdenum is less than 5 ppb by mass.

The container for filling the acid fluoride is a manganese steel (STH12) cylinder (manufactured by Asahi Glassplant Inc.) having a capacity of 10 L. The surface roughness Rz of the inner surface of the cylinder is 5 μm.

Using a filling device, the carbonyl fluoride was supplied from a raw material tank to a cylinder under the conditions of 3 MPa and 50° C. and then sealed. After being allowed to stand for 2 hours after the sealing (treatment step), the entire amount of the carbonyl fluoride was extracted from the cylinder (extraction step).

Next, using a filling device, 5 kg of carbonyl fluoride was supplied from the raw material tank to the cylinder after the extraction step was carried out, and the cylinder was sealed (filling step). Then, the cylinder was stored under a temperature condition of 30° C. to 35° C. for 12 months.

A part of the carbonyl fluoride was extracted from the cylinder after being stored for 12 months. Then, the concentration of gas impurities (carbon monoxide, carbon dioxide, carbon tetrafluoride, and hydrogen fluoride) in the carbonyl fluoride was measured using a Fourier transform infrared spectrometer MAX-iR manufactured by Thermo Fisher Scientific, Inc. In addition, the concentrations of metal impurities (sodium, chromium, manganese, iron, cobalt, nickel, copper, and molybdenum) in the liquid phase of the carbonyl fluoride were measured using an inductively coupled plasma mass spectrometer Element 2 manufactured by Thermo Fisher Scientific, Inc. The results are shown in Table 2.

The measurement conditions for the gas impurities are as follows. Gas impurities in the carbonyl fluoride accommodated in the raw material tank are also measured under the same conditions.
Measurement temperature: 27° C.
Measurement pressure: 101.3 to 102 kPa
Cell length: 10 m
Cell window plate: AgCl
Measurement wavelength:
Carbon monoxide: 2172 cm⁻¹
Carbon dioxide: 2360 cm⁻¹
Hydrogen fluoride: 4038 cm⁻¹
Carbon tetrafluoride: 1281 cm⁻¹

The measurement conditions for the metal impurities are as follows. Metal impurities in the carbonyl fluoride accommodated in the raw material tank are also measured under the same conditions.

First, a liquid phase of the carbonyl fluoride was extracted from the cylinder and vaporized, and then the vaporized carbonyl fluoride was subjected to bubbling in 100 g of an aqueous solution having a concentration of 1% by mass of nitric acid. As a result, a mixed liquid of carbonyl fluoride and an aqueous solution of nitric acid was prepared. Since the mass of the mixed liquid was 200 g, it was considered that 100 g of carbonyl fluoride was mixed with 100 g of the aqueous solution of nitric acid.

Next, 1 g of the mixed liquid was extracted, the metal was analyzed using an inductively coupled plasma mass spectrometer, and the signal intensity (y) of each of sodium, chromium, manganese, iron, cobalt, nickel, copper, and molybdenum in the mixed liquid was measured. Then, using a calibration curve, the concentration of each metal was calculated from the signal intensity.

The calibration curve was created as follows. More specifically, standard solutions of nitric acid, which respectively had metal concentrations of 0 mass ppb (not containing a metal), 10 mass ppb, and 100 mass ppb, were produced, and the analysis was carried out using an inductively coupled plasma mass spectrometer. Then, the calibration curve was created so that the concentration of the metal was plotted on the horizontal axis and the signal intensity was plotted on the vertical axis, and the slope (a) and the intercept (b) thereof were determined. The same operation was carried out for sodium, chromium, manganese, iron, cobalt, nickel, copper, and molybdenum to create a calibration curve for each metal.

The concentration M of the metal contained in the mixed liquid can be calculated according to the following expression.

M=(y−b)/a

TABLE 1

| | Container | | Treatment step | | | | Extraction step | | Filling step | |
|---|---|---|---|---|---|---|---|---|---|---|
| | | Surface | Acid | Pres- | Temp- | | | Number | | Filling |
| | Mater- | roughness | fluoride for | sure | erature | Time | Extraction | of times | | amount |
| | ial | Rz (um) | treatment | (MPa) | (° C.) | (h) | amount | (times) | Acid fluoride | (kg) |
| Ex. 1 | STH12 | 5 | Carbonyl | 3 | 50 | 2 | Entire | 1 | Carbonyl | 5 |
| | | | fluoride | | | | amount | | fluoride | |
| Ex. 2 | STH12 | 5 | Oxalyl | 0.15 | 50 | 2 | Entire | 1 | Oxalyl | 5 |
| | | | fluoride | | | | amount | | fluoride | |
| Ex. 3 | STH12 | 5 | Trifluoromethyl | 3 | 50 | 2 | Entire | 1 | Trifluoromethyl | 5 |
| | | | acetyl fluoride | | | | amount | | acetyl fluoride | |
| Ex. 4 | STH21 | 5 | Carbonyl | 3 | 50 | 2 | Entire | 1 | Carbonyl f | 5 |
| | | | fluoride | | | | amount | | luoride | |
| Ex. 5 | STH21 | 5 | Oxalyl | 0.15 | 50 | 2 | Entire | 1 | Oxalyl | 5 |
| | | | fluoride | | | | amount | | fluoride | |
| Ex. 6 | STH21 | 5 | Trifluoromethyl | 3 | 50 | 2 | Entire | 1 | Trifluoromethyl | 5 |
| | | | acetyl fluoride | | | | amount | | acetyl fluoride | |
| Ex. 7 | STH12 | 10 | Carbonyl | 3 | 50 | 2 | Entire | 1 | Carbonyl | 5 |
| | | | fluoride | | | | amount | | fluoride | |
| Ex. 8 | STH12 | 10 | Carbonyl | 3 | 50 | 2 | Entire | 3 | Carbonyl | 5 |
| | | | fluoride | | | | amount | | fluoride | |
| Ex. 9 | STH12 | 2 | Carbonyl | 3 | 50 | 2 | Entire | 1 | Carbonyl | 5 |
| | | | fluoride | | | | amount | | fluoride | |
| Ex. 10 | STH12 | 5 | Carbonyl | 3 | 20 | 48 | Entire | 1 | Carbonyl | 5 |
| | | | fluoride | | | | amount | | fluoride | |
| Ex. 11 | STH12 | 5 | Carbonyl | 3 | 160 | 1 | Entire | 1 | Carbonyl f | 5 |
| | | | fluoride | | | | amount | | luoride | |
| Ex. 12 | STH12 | 5 | Carbonyl | 3 | 50 | 2 | 10% | 1 | Carbonyl | 5 |
| | | | fluoride | | | | | | fluoride | |
| Ex. 13 | STH12 | 5 | Carbonyl | 3 | 50 | 2 | Entire | 1 | Carbonyl | 1 |
| | | | fluoride | | | | amount | | fluoride | |
| Ex. 14 | STH12 | 5 | Carbonyl | 3 | 50 | 2 | Entire | 1 | Trifluoromethyl | 5 |
| | | | fluoride | | | | amount | | acetyl fluoride | |
| Comp. | STH12 | 5 | lodine | 0.7 | 50 | 2 | Entire | 1 | lodine | 5 |
| Ex. 1 | | | heptafluoride | | | | amount | | heptafluoride | |
| Comp. | STH12 | 15 | Carbonyl | 3 | 50 | 2 | Entire | 1 | Carbonyl f | 5 |
| Ex. 2 | | | fluoride | | | | amount | | luoride | |
| Comp. | STH12 | 5 | Carbonyl | — | — | — | — | — | Carbonyl | 5 |
| Ex. 3 | | | fluoride | | | | | | fluoride | |
| Comp. | STH12 | 5 | Carbonyl | 3 | 170 | 2 | Entire | 1 | Carbonyl f | 5 |
| Ex. 4 | | | fluoride | | | | amount | | luoride | |

TABLE 2

| | HF | CO₂ | CO | CF₄ | Na | Cr | Mn | Fe | Co | Ni | Cu | Mo |
|---|---|---|---|---|---|---|---|---|---|---|---|---|
| Ex. 1 | 3 | 2 | 3 | 3 | Less | Less | Less | Less | Less | Less | Less | Less |
| | | | | | than 5 | than 5 | than 5 | than 5 | than 5 | than 5 | than 5 | than 5 |
| Ex. 2 | 3 | 3 | 2 | 3 | Less | Less | Less | Less | Less | Less | Less | Less |
| | | | | | than 5 | than 5 | than 5 | than 5 | than 5 | than 5 | than 5 | than 5 |
| Ex. 3 | 3 | 5 | 3 | 3 | Less | Less | Less | Less | Less | Less | Less | Less |
| | | | | | than 5 | than 5 | than 5 | than 5 | than 5 | than 5 | than 5 | than 5 |
| Ex. 4 | 2 | 2 | 2 | Less | Less | Less | Less | Less | Less | Less | Less | Less |
| | | | | than 2 | than 5 | than 5 | than 5 | than 5 | than 5 | than 5 | than 5 | than 5 |
| Ex. 5 | 2 | 3 | 5 | 5 | Less | Less | Less | Less | Less | Less | Less | Less |
| | | | | | than 5 | than 5 | than 5 | than 5 | than 5 | than 5 | than 5 | than 5 |
| Ex. 6 | 3 | 3 | 3 | 2 | Less | Less | Less | Less | Less | Less | Less | Less |
| | | | | | than 5 | than 5 | than 5 | than 5 | than 5 | than 5 | than 5 | than 5 |
| Ex. 7 | 4 | 5 | 4 | 3 | Less | Less | Less | Less | Less | Less | Less | Less |
| | | | | | than 5 | than 5 | than 5 | than 5 | than 5 | than 5 | than 5 | than 5 |
| Ex. 8 | 2 | 3 | 2 | 3 | Less | Less | Less t | Less | Less | Less | Less | Less |
| | | | | | than 5 | than 5 | han 5 | than 5 | than 5 | than 5 | than 5 | than 5 |
| Ex. 9 | 2 | 2 | 3 | 2 | Less | Less | Less | Less | Less | Less | Less | Less |
| | | | | | than 5 | than 5 | than 5 | than 5 | than 5 | than 5 | than 5 | than 5 |
| Ex. 10 | 10 | 10 | 5 | 6 | Less | Less | Less | Less | Less | Less | Less | Less |
| | | | | | than 5 | than 5 | than 5 | than 5 | than 5 | than 5 | than 5 | than 5 |
| Ex. 11 | 9 | 10 | 8 | 8 | Less | Less | Less | Less | Less | Less | Less | Less |
| | | | | | than 5 | than 5 | than 5 | than 5 | than 5 | than 5 | than 5 | than 5 |
| Ex. 12 | 7 | 8 | 7 | 5 | Less | Less | Less | Less | Less | Less | Less | Less |
| | | | | | than 5 | than 5 | than 5 | than 5 | than 5 | than 5 | than 5 | than 5 |
| Ex. 13 | 4 | 6 | 5 | 3 | Less | Less | Less | Less | Less | Less | Less | Less |
| | | | | | than 5 | than 5 | than 5 | than 5 | than 5 | than 5 | than 5 | than 5 |
| Ex. 14 | 3 | 2 | 3 | 3 | Less | Less | Less | Less | Less | Less | Less | Less |
| | | | | | than 5 | than 5 | than 5 | than 5 | than 5 | than 5 | than 5 | than 5 |
| Comp. | 21 | 37 | 13 | 52 | 7 | 118 | 7 | 85 | 8 | 6 | 13 | 21 |
| Ex. 1 | | | | | | | | | | | | |
| Comp. | 18 | 24 | 11 | 36 | 8 | 121 | 8 | 31 | 6 | 7 | 8 | 6 |
| Ex. 2 | | | | | | | | | | | | |
| Comp. | 4 | 37 | 41 | 4 | Less | 232 | Less | Less | Less | Less | Less | Less |
| Ex. 3 | | | | | than 5 | | than 5 | than 5 | than 5 | than 5 | than 5 | than 5 |
| Comp. | 27 | 31 | 22 | 17 | 7 | 109 | 12 | 35 | 15 | 20 | 10 | 8 |
| Ex. 4 | | | | | | | | | | | | |

*) The unit of the numerical value is ppm by volume for gases, and ppb by mass for metals.

### Examples 2 to 14 and Comparative Examples 1, 2, and 4

An acid fluoride-filled container was produced in the same manner as in Example 1, except that the material of the cylinder (container), the surface roughness Rz of the inner surface of the cylinder, the type of the acid fluoride for treatment, the conditions for the treatment step (pressure, temperature, and time), the conditions for the extraction step (the extraction amount, the number of times of the extraction step carried out), the type of the acid fluoride filled into the cylinder, and the conditions for the filling step (filling amount) were changed as shown in Table 1. The number of times of the extraction step carried out means the number of cycles in which the extraction step is repeatedly carried out when one cycle is defined as carrying out the treatment step and the extraction step continuously.

Then, each of the concentrations of the gas impurities and the metal impurities in the acid fluoride was measured in the same manner as in Example 1. The results are shown in Table 2.

Gas impurities and metal impurities, which are contained in the oxalyl fluoride accommodated in the raw material tank, are as follows. More specifically, the content of carbon monoxide is 6 ppm by volume, the content of carbon dioxide is 3 ppm by volume, the content of hydrogen fluoride is 3 ppm by volume, the content of carbon tetrafluoride is 5 ppm by volume, the content of sodium is less than 5 ppb by mass, the content of chromium is less than 5 ppb by mass, the content of manganese is less than 5 ppb by mass, the content of iron is less than 5 ppb by mass, the content of cobalt is less than 5 ppb by mass, the content of nickel is less than 5 ppb by mass, the content of copper is less than 5 ppb by mass, and the content of molybdenum is less than 5 ppb by mass.

In addition, gas impurities and metal impurities, which are contained in trifluoromethyl acetyl fluoride accommodated in the raw material tank, are as follows. More specifically, the content of carbon monoxide is 3 ppm by volume, the content of carbon dioxide is 5 ppm by volume, the content of hydrogen fluoride is 4 ppm by volume, the content of carbon tetrafluoride is 3 ppm by volume, the content of sodium is less than 5 ppb by mass, the content of chromium is less than 5 ppb by mass, the content of manganese is less than 5 ppb by mass, the content of iron is less than 5 ppb by mass, the content of cobalt is less than 5 ppb by mass, the content of nickel is less than 5 ppb by mass, the content of copper is less than 5 ppb by mass, and the content of molybdenum is less than 5 ppb by mass.

Further, gas impurities and metal impurities, which are contained in the iodine heptafluoride accommodated in the raw material tank, are as follows. More specifically, the content of carbon monoxide is 2 ppm by volume, the content of carbon dioxide is 2 ppm by volume, the content of hydrogen fluoride is 2 ppm by volume, the content of carbon tetrafluoride is less than 1 ppm by mass, the content of sodium is less than 5 ppb by mass, the content of chromium is less than 5 ppb by mass, the content of manganese is less than 5 ppb by mass, the content of iron is less than 5 ppb by mass, the content of cobalt is less than 5 ppb by mass, the content of nickel is less than 5 ppb by mass, the content of copper is less than 5 ppb by mass, and the content of molybdenum is less than 5 ppb by mass.

### Comparative Example 3

An acid fluoride-filled container was produced in the same manner as in Example 1, except that the filling step was carried out without carrying out the treatment step and the extraction step. Then, each of the concentrations of the gas impurities and the metal impurities in the acid fluoride was measured in the same manner as in Example 1. The results are shown in Table 2.

The following can be seen from the results of Examples 1 to 3. When an acid fluoride-filled container was produced by using carbonyl fluoride, oxalyl fluoride, or trifluoromethyl acetyl fluoride as the acid fluoride in the treatment step and the filling step and using manganese steel as a material of the container for being filled with the acid fluoride, the amounts of gas impurities and metal impurities were not increased even when the acid fluoride was stored in the container for a certain period of time. In particular, when oxalyl fluoride was used as the acid fluoride for treatment, it was possible to produce an acid fluoride-filled container without any problem even when the pressure condition of the treatment step was 0.15 MPa.

The following can be seen from the results of Examples 4 to 6. It was possible to produce an acid fluoride-filled container without any problem even when chromium-molybdenum steel was used as a material of the container for being filled with the acid fluoride. In addition, the amounts of gas impurities and metal impurities were not increased even when the acid fluoride was stored in the container for a certain period of time.

The following can be seen from the results of Examples 7 to 9. It was possible to produce an acid fluoride-filled container without any problem even when the surface roughness Rz of the inner surface of the container was 10 μm or 2 μm. In addition, the amounts of gas impurities and metal impurities were hardly increased even when the acid fluoride was stored in the container for a certain period of time. In particular, when the treatment step and the extraction step were repeated for three cycles and when the surface roughness Rz of the inner surface of the container was reduced (2 μm), the effect of suppressing an increase in the amounts of gas impurities and metal impurities was high.

The following can be seen from the results of Example 10 and 11. It was possible to produce an acid fluoride-filled container without any problem even when the temperature of the treatment step was set to 20° C. or 160° C. In addition, the amounts of gas impurities and metal impurities were not significantly increased even when the acid fluoride was stored in the container for a certain period of time.

The following can be seen from the result of Example 12. It was possible to produce an acid fluoride-filled container without any problem even when only 10% of the amount of the carbonyl fluoride introduced into the container in the treatment step was extracted in the extraction step, and then the filling step was carried out. In addition, even when the acid fluoride was stored in the container for a certain period of time, the amount of impurities was not significantly increased.

The following can be seen from the result of Example 13. It was possible to produce an acid fluoride-filled container without any problem even when the filling amount of the acid fluoride to be filled into the container in the filling step was set to 1 kg. In addition, the amounts of gas impurities and metal impurities were not increased even when the acid fluoride was stored in the container for a certain period of time.

The following can be seen from the result of Example 14. It was possible to produce an acid fluoride-filled container without any problem even when different types of acid fluorides were used in the treatment step and the filling step. In addition, the amounts of gas impurities and metal impurities were not increased even when the acid fluoride was stored in the container for a certain period of time.

The following can be seen from the result of Comparative Example 1. When iodine heptafluoride was used instead of the acid fluoride, the amounts of gas impurities and metal impurities were increased after iodine heptafluoride was stored in the container for a certain period of time. In particular, since the hydrogen fluoride was significantly increased to 100 ppm by volume or more and the chromium was significantly increased to 100 ppb by volume or more, it was suggested that the present disclosure is a method for producing a filled container which is effective for an acid fluoride.

The following can be seen from the result of Comparative Example 2. Even when the material of the container was manganese steel, the amounts of gas impurities and metal impurities were increased after the acid fluoride was stored in the container for a certain period of time, when the surface roughness Rz of the inner surface of the container was large. From this result, it was suggested that the condition of the surface roughness Rz of the inner surface of the container is important.

The following can be seen from the result of Comparative Example 3. When the container that has not carried out the treatment step and the extraction step is filled with the acid fluoride, the amounts of gas impurities and metal impurities were increased after the acid fluoride was stored in the container for a certain period of time. From this result, it was suggested that it is essential to carry out the treatment step and the extraction step. The following can be seen from the result of Comparative Example 4. When the temperature of the treatment step exceeded 160° C., the amounts of gas impurities and metal impurities were increased after the acid fluoride was stored in the container for a certain period of time. From this result, it was suggested that the temperature condition of the treatment step is important.

### REFERENCE SIGNS LIST

1: raw material tank
2: cylinder
3: pipe
5: branch pipe

1. A method for producing an acid fluoride-filled container in which an acid fluoride is filled into a container, wherein at least an inner surface of the container is formed of a metal material and a surface roughness Rz of the inner surface is 0.01 μm or more and 10 μm or less, and the method comprising:
a treatment step of introducing a treatment gas containing an acid fluoride for treatment for treating the inner surface, into the container to bring the inner surface into contact with the acid fluoride for treatment at a temperature of 160° C. or less;
an extraction step of extracting the treatment gas introduced into the container in the treatment step, from the container; and
a filling step of filling the container after the extraction step with an acid fluoride for being stored in the container.

2. The method for producing an acid fluoride-filled container according to claim 1, wherein in the metal material forming the inner surface, an iron content is 60% by mass or more, and a chromium content is 10% by mass or less.

3. The method for producing an acid fluoride-filled container according to claim 1, wherein the metal material forming the inner surface is at least one of manganese steel and chromium-molybdenum steel.

4. The method for producing an acid fluoride-filled container according to claim 1, wherein a content of any of carbon monoxide, carbon dioxide, carbon tetrafluoride, and hydrogen fluoride in the acid fluoride extracted from the container after the filling step is 10 ppm by volume or less.

5. The method for producing an acid fluoride-filled container according to claim 1, wherein a content of any metal contained in a liquid phase of the acid fluoride extracted from the container after the filling step is less than 100 ppb by mass for any metal.

6. The method for producing an acid fluoride-filled container according to claim 1, wherein the acid fluoride filled into the container in the filling step is at least one of carbonyl fluoride, oxalyl fluoride, and trifluoromethyl acetyl fluoride.

7. The method for producing an acid fluoride-filled container according to claim 2, wherein a content of any of carbon monoxide, carbon dioxide, carbon tetrafluoride, and hydrogen fluoride in the acid fluoride extracted from the container after the filling step is 10 ppm by volume or less.

8. The method for producing an acid fluoride-filled container according to claim 3, wherein a content of any of carbon monoxide, carbon dioxide, carbon tetrafluoride, and hydrogen fluoride in the acid fluoride extracted from the container after the filling step is 10 ppm by volume or less.

9. The method for producing an acid fluoride-filled container according to claim 2, wherein a content of any metal contained in a liquid phase of the acid fluoride extracted from the container after the filling step is less than 100 ppb by mass for any metal.

10. The method for producing an acid fluoride-filled container according to claim 3, wherein a content of any metal contained in a liquid phase of the acid fluoride extracted from the container after the filling step is less than 100 ppb by mass for any metal.

11. The method for producing an acid fluoride-filled container according to claim 2, wherein the acid fluoride filled into the container in the filling step is at least one of carbonyl fluoride, oxalyl fluoride, and trifluoromethyl acetyl fluoride.

12. The method for producing an acid fluoride-filled container according to claim 3, wherein the acid fluoride filled into the container in the filling step is at least one of carbonyl fluoride, oxalyl fluoride, and trifluoromethyl acetyl fluoride.
